(19) United States
(12) Patent Application Publication
(10) Pub. No.: US 2013/0171697 A1
(43) Pub. Date: Jul. 4, 2013

(54) MICROFLUIDIC DEVICE COMPRISING ROTATABLE DISC-TYPE BODY, AND METHODS OF SEPARATING TARGET MATERIAL AND AMPLIFYING NUCLEIC ACID USING THE SAME

(71) Applicant: Samsung Electronics Co., Ltd., Suwon-si (KR)

(72) Inventors: Jong-myeon PARK, Incheon (KR); Myoung-soon KIM, Seoul (KR); Ye-ryoung YONG, Seoul (KR)

(73) Assignee: Samsung Electronics Co., Ltd., Suwon-si (KR)

(21) Appl. No.: 13/662,333

(22) Filed: Oct. 26, 2012

(30) Foreign Application Priority Data
Jan. 4, 2012 (KR) 10-2012-0001145

(51) Int. Cl. C12P 19/34; C07H 21/00; B01L 3/00

(52) U.S. Cl. 435/91.2; 422/506; 536/25.41

(57) ABSTRACT

A microfluidic device for controlling a flow of a fluid using centrifugal and rotational force based on a rotatable disc-type body, a method of separating a target material or performing emulsion nucleic acid amplification using the microfluidic device.

### CROSS-REFERENCE TO RELATED APPLICATIONS

This application claims the benefit of Korean Patent Application No. 10-2012-0001145, filed on Jan. 4, 2012, in the Korean Intellectual Property Office, the disclosures of which are incorporated herein in their entireties by reference.

### BACKGROUND

1. Field

The present disclosure relates to a microfluidic device capable of controlling a flow of a fluid, and methods of separating a target material and amplifying a nucleic acid using the same.

2. Description of the Related Art

Techniques for separating a target material from a sample by using biochemical reactions are widely used in various fields, including the medical field. However, complicated and repetitive processes need to be manually carried out to perform most biochemical reactions. Accordingly, it is difficult to obtain reliable results due to mistakes and errors occurring during the processes. For example, a technique for separating a genome from whole blood includes complicated manual operations such as using a plurality of reagents, mixing the reagents with a sample, and centrifuging the mixture in order to separate white blood cells from the whole blood, thereby separating the genome from the white blood cells. In another example, in order to perform an emulsion nucleic acid amplification that includes preparing an emulsion of a template nucleic acid sample, a reagent for a nucleic acid amplification, and the like, heating and cooling the emulsion, decomposing the emulsion, and various mixing and centrifuging operations are required. Additionally, a variety of devices for these operations need to be used. Thus, in order to quickly obtain accurate results for separation of a target material from a sample by using various biochemical reactions, there is a need to develop a device that efficiently and automatically mixes a sample and a reagent, centrifuges the mixture, and the like.

### SUMMARY

Provided is a microfluidic device for controlling a flow of a fluid using centrifugal force and rotational force based on a rotatable disc-type body, a method of separating a target material using the device, and an emulsion nucleic acid amplification method using the microfluidic device.

Additional aspects will be set forth in part in the description which follows and, in part, will be apparent from the description, or may be learned by practice of the presented embodiments.

According to an aspect of the present invention, a microfluidic device capable of controlling a flow of a fluid for separating a target material from a mixture of two or more materials with different densities using centrifugal and rotational force, includes a rotatable disc-type body; a first chamber that is disposed in or on the body and spaced apart from a center of the body in a centrifugal force direction (e.g., a direction away from the rotational center of the body and towards the periphery of the body); a second chamber that is disposed to be spaced apart from the first chamber in the centrifugal force direction and in fluid communication with the first chamber, wherein the second chamber comprises an upper outlet and a lower outlet spaced apart from the upper outlet in the centrifugal force direction; and a plurality of third chambers in fluid communication with the upper outlet and the lower outlet of the second chamber, respectively.

According to another aspect of the present invention, a method of separating a target material using the microfluidic device is provided. The method comprises introducing at least one reagent to the first chamber; introducing a sample comprising a target material to the second chamber; rotating the body of the device, thereby transferring the reagent from the first chamber to the second chamber and mixing and reacting the reagent with the sample, wherein the body of the device is optionally rotated repeatedly in a clockwise or counterclockwise direction, or sequentially in the clockwise and counterclockwise direction. The method further comprises centrifuging products of the reagent-sample reaction by continuously rotating the body, for instance, continuously rotating in a clockwise or counter-clockwise direction; and separating the target material contained in a upper layer or lower layer of the second chamber via an upper outlet or a lower outlet of the second chamber.

According to another aspect of the present invention, a method of amplifying a nucleic acid using the microfluidic device is provided. The method comprises providing a microfluidic device, as described above, comprising a plurality of first chambers in fluid communication with a second chamber; introducing at least one nucleic acid amplification reagent to at least one of the plurality of first chambers, and at least one oil used to perform a nucleic acid amplification to at least one other of the plurality of first chambers of the device; introducing a sample including a template nucleic acid to the second chamber of the device; rotating the body of the device, thereby transferring the reagent from one first chamber to the second chamber and mixing the reagent with the sample, and transferring the oil from another first chamber to the second chamber by rotating the body and forming an emulsion in which the sample is surrounded by the oil; wherein the body of the device is optionally repeatedly rotated in a clockwise or counterclockwise direction, or sequentially rotated in a clockwise and counterclockwise direction; amplifying a nucleic acid of the sample in the emulsion by heating and/or cooling the second chamber; centrifuging the resultant by continuously rotating the body, for instance, in one of a clockwise or counter-clockwise direction; and separating products of the amplification contained in a supernatant or a sediment via an upper outlet or a lower outlet of the second chamber.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and/or other aspects will become apparent and more readily appreciated from the following description of the embodiments, taken in conjunction with the accompanying drawings of which:

FIG. 1 schematically shows a rotatable disc-type body that performs a biochemical reaction using a centrifugal force and a rotational force;

FIG. 2 shows a microfluidic device according to an embodiment of the present disclosure;

FIG. 3 shows a fourth chamber in fluid communication with a third chamber in a microfluidic device according to an embodiment of the present disclosure;

FIG. 4 shows a heater and a cooler of a microfluidic device according to an embodiment of the present disclosure;

FIG. 5 shows a series of operations of a biochemical reaction conducted by using a microfluidic device according to an embodiment of the present disclosure;

FIG. 6 shows a series of operations for separating white blood cells from a whole blood sample using a microfluidic device according to an embodiment of the present disclosure; and

FIG. 7 shows a series of operations for controlling transfers of a sample and a reagent according to an embodiment of the present disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

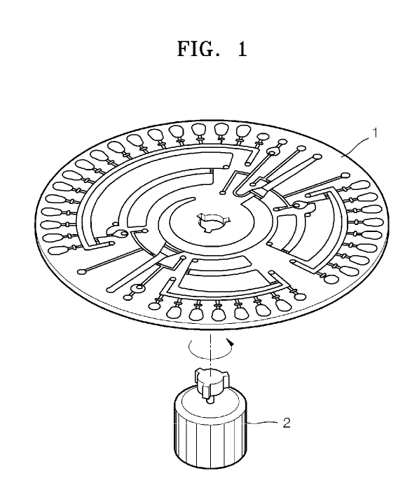

FIG. 1 schematically shows a rotatable disc-type body that performs a biochemical reaction using a centrifugal force and a rotational force.

A microfluidic device according to an embodiment of the present disclosure includes a rotatable disc-type body 1 that is mounted on a rotatable drive shaft 2 and controls a flow of a fluid by applying a centrifugal force and a rotational force thereto. The microfluidic device includes an ultra-small device that accommodates, for instance, a fluid having a volume less than a microliter (μl), and has a width, a length, and a height each in a range of about 0.1 μm to 20 mm, so that a flow of the fluid is controllable. Referring to FIG. 1, the microfluidic device may include at least one chamber in which various fluids are accommodated and biochemical reactions are performed, a channel that connects the chambers so as to allow a fluid to flow therebetween, and a valve that controls a flow of a fluid in the chambers and the channel. Accordingly, the microfluidic device may include various chambers, channels, and valves for controlling a flow of a fluid. Particularly, the microfluidic device may include channels used to flow a fluid between a first chamber and a second chamber and between the second chamber and a third chamber, and valves for opening or closing the channel, thereby allowing flow of the fluid or stopping the flow of the fluid. In this regard, the valves may vary within a known range. For example, the valves may include a phase transition type material, for example, the phase of which changes by energy of electromagnetic waves. The phase transition material may be wax or gel. The valves may include exothermic microparticles that are dispersed in the phase transition material and absorb energy of electromagnetic waves to emit heat. The exothermic microparticles may be metal oxide, polymer particles, quantum dots, or magnetic beads formed of Al₂O₃, TiO₂, Ta₂O₃, Fe₂O₃, Fe₃O₄, or HfO₂. The exothermic microparticles may vary according to the size of the channels. Meanwhile, the microfluidic device may further include any other element for controlling a flow of a fluid known in the art

The microfluidic device includes the rotatable disc-type body 1 as shown in FIG. 1. The rotatable disc-type body 1 rotates with respect to the rotatable drive shaft 2 to transfer a fluid from one chamber to another chamber based on a centrifugal force and a rotational force applied to the fluid accommodated in one of the chambers, and may not require a separate pump for providing a driving force for the transfer of the fluid. A fluid may flow through the channels, and opening and closing of the channels may be controlled by the valves. Thus, the microfluidic device includes at least two chambers radially aligned with respect to the center of the body 1. Meanwhile, the rotatable drive shaft 2 may be connected to a unit for providing a rotational force, for example, a motor or servo motor, and accordingly the body 1 may rotate in the clockwise or counter-clockwise direction.

The microfluidic device may include a fluidic control system that includes a program for encoding continuous instructions on a start of rotation and a rotational direction of the rotatable drive shaft 2 and opening and closing of the valves, or may be connected in an operable manner to the fluidic control system. The program and/or instructions may be stored on a non-transient, computer readable medium. Accordingly, the microfluidic device may control a flow of a fluid by controlling a rotational force on the body 1, a centrifugal force generated thereby, and operations of the valves using the fluidic control system connected to a user's terminal.

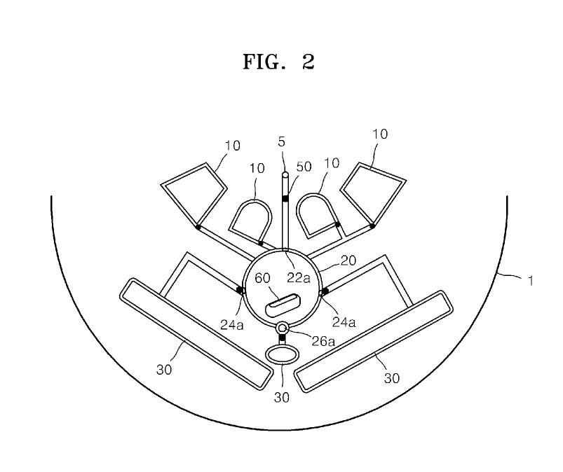

FIG. 2 shows a microfluidic device according to an embodiment of the present disclosure.

The microfluidic device according to the current embodiment includes a rotatable disc-type body 1, a first chamber 10 that is disposed to be spaced apart from a center 5 of the rotatable disc-type body 1 in a centrifugal force direction. The microfluidic device of this embodiment also includes a second chamber 20 that is disposed to be spaced apart from the first chamber 10 in the centrifugal force direction and in fluid communication with the first chamber, and includes an upper outlet 24a and lower outlet(s) 26a spaced apart from the upper outlet 24a in the centrifugal force direction. This embodiment also includes third chambers 30 that are in fluid communication with both of the upper outlet 24a and the lower outlet(s) 26a of the second chamber 20. The various chambers and/or outlets may be in fluid communication with one another by way of fluid communicating passages connecting the chambers and or outlets. Furthermore, the fluid communicating passages may comprise one or more valves to control the flow of fluid.

The rotatable disc-type body 1 is described above.

The first chamber 10 is a chamber for containing at least one reagent and/or sample for a biochemical reaction. The first chamber 10 may be fabricated according to various standards. For example, the first chamber 10 may have volumes and shapes corresponding to the type and amount of the reagent and/or sample. The reagent may include any material used to perform a biochemical reaction, and the sample may include any material including a target material to be identified by the biochemical reaction. For example, in an extraction of a nucleic acid from a cell, the reagent may include a cell lysing solution, a cell purifying solution, a nucleic acid concentrating solution, a washing solution, a buffer, distilled water, and/or a cell eluting solution. The sample may include whole blood including a target nucleic acid. In another example, in an emulsion nucleic acid amplification, the reagent may include oil used to form an external layer in the formation of an emulsion, a forward primer, a reverse primer, dNTPs (dATP, dCTP, dGTP, and dTTP), a nucleic acid polymerase (e.g., Taq polymerase), and a buffer used to a perform polymerase chain reaction (PCR). The sample may be a solution including a template nucleic acid such as deoxyribonucleic acid (DNA), ribonucleic acid (RNA), peptide nucleic acid (PNA), and locked nucleic acid (LNA). Thus, the first chamber 10 of the microfluidic device may include at least one reagent used to extract a genome from a cell, or a reagent used to amplify a target region of a genome. In addition, the first chamber 10 may be prepared by sealing a chamber already including a reagent and/or a sample, or by preparing a chamber with an inlet and a cover thereof, adding a reagent and/or a sample, and sealing the chamber. The first chamber 10 is spaced apart from the center 5 of the body 1 in the centrifugal force direction. For instance, the first chamber 10 is disposed in or on the body at a position spaced apart from the rotational center of the body, in a direction in which a centrifugal force is applied when the body is rotated. The first chamber, or plurality of first chambers, may be arranged to form a concentric circle in a circumferential direction relative to the rotational center of the body.

Referring to FIG. 2, four first chambers 10 are aligned and separated from each other with respect to the center 5 of the body 1 in the centrifugal force direction to form a concentric circle. In this regard, the first chamber 10 may further include a valve 50 capable of controlling the opening and closing of the fluid path, thereby allowing a flow of a fluid and stopping a flow of a fluid, at a portion connected to the second chamber 20 in a fluid communication manner. In some cases, the first chamber 10 contains the reagent and/or sample. If the body 1 rotates when the valve 50 is open, a driving force is applied to the first chamber 10 in a direction of a resultant force of a rotational force and a centrifugal force, and thus the reagent and/or sample accommodated in the first chamber 10 may flow to the second chamber 20. In addition, the amount of the reagent and/or sample flowing from the first chamber 10 to the second chamber 20 may be controlled by controlling the rotation speed of the body 1 and/or by controlling opening and closing of the valve 50. In other cases, the first chamber 10 includes the reagent of the biochemical reaction, and the sample of the biochemical reaction may be directly added to the second chamber 20 through a separate inlet 22a. Referring to FIG. 2, the second chamber 20 includes an inlet 22a different from that of the first chamber 10. In this regard, the first chamber 10 includes the reagent of the biochemical reaction, and the sample of the biochemical reaction is introduced into the second chamber 20 via the inlet 22a.

In the second chamber 20, the reagent and/or sample introduced from the first chamber 10 are mixed and/or the biochemical reaction is performed. The second chamber 20 is aligned to be spaced apart from the first chamber 10 in the centrifugal force direction and in fluid communication with the first chamber(s). In this regard, the second chamber 20 is aligned in a direction in which a centrifugal force is applied with respect to the concentric circle of the first chamber 10, i.e., the second chamber(s) can be arranged to form another concentric circle in the circumferential direction, so as to allow a fluid to flow between the first chamber 10 and the second chamber 20. The first chamber 10 may be connected to the second chamber 20 via one of the channels having the valve 50.

Referring to FIG. 2, one second chamber 20 is disposed on a concentric circle different from that of the first chamber 10 in the centrifugal force direction. In addition, the second chamber 20 includes the upper outlet 24a and the lower outlet 26a aligned to be spaced apart from the upper outlet 24a in the centrifugal force direction. The upper outlet 24a and the lower outlet 26a are used to separate at least two material layers formed in the second chamber 20 before or after the reaction. The upper outlet 24a and the lower outlet 26a may further be disposed in various desired positions in the second chamber 20. For example, referring to FIG. 2, two upper outlets 24a are symmetrically disposed at two positions of the circumference of the second chamber 20, and one lower outlet 26a is disposed at a terminal to be spaced apart from the upper outlets 24a in the centrifugal force direction in the second chamber 20. In this regard, the valve 50 may be installed in the upper outlet 24a and the lower outlet 26a. Thus, when the second chamber 20 includes the reagent and/or sample, and the mixture is separated to at least two layers before or after the reaction of the reagent and/or sample, if the body 1 is rotated while the valve 50 of the upper outlet 24a is open and the valve 50 of the lower outlet 26a is closed, a driving force is applied to the second chamber 20 in a direction of a resultant force of a rotational force and a centrifugal force. Then, the upper layer (for example, a supernatant) of the second chamber 20 flows into the third chamber 30 connected to the upper outlet 24a in a fluid communication manner. In addition, the amount of the upper layer (for example, a supernatant) flowing from the second chamber 20 to the third chamber 30 connected to the upper outlet 24a of the second chamber 20 in a fluid communication manner may also be controlled by controlling the rotation speed of the body 1 and/or by controlling opening and closing of the valve 50. In addition, when the second chamber 20 includes the reagent and/or sample, and the mixture is separated into at least two layers before or after the reaction of the reagent and/or sample, if the body 1 is rotated while the valve 50 of the upper outlet 24a is closed and the valve 50 of the lower outlet 26a is open, a driving force is applied to the second chamber 20 in a direction of a resultant force of a rotational force and a centrifugal force. Then, the lower layer (for example, a sediment or a precipitate) of the second chamber 20 flows into a third chamber 30 connected to the lower outlet 26a in a fluid communication manner. In addition, the amount of the lower layer flowing from the second chamber 20 to the third chamber 30 connected to the lower outlet 26a of the second chamber 20 in a fluid communication manner may also be controlled by controlling the rotation speed of the body 1 and/or by controlling opening and closing of the valve 50.

The third chambers 30 are respectively connected to the upper outlet 24a and the lower outlet 26a of the second channel 20 in a fluid communication manner. As described above, materials of at least two layers separated in the second channel 20 due to density difference may be respectively collected in the third chambers 30. The third chambers 30 connected to the upper outlet 24a and the lower outlet 26a of the second channel 20 in a fluid communication manner may be spaced apart from the second chamber 20 in the centrifugal force direction in a fluid communication manner. The third chamber 30 may be fabricated according to various standards. For example, the chamber 30 may have volumes and shapes corresponding to the type of materials introduced from the second chamber 20, and two or more third chambers 30 may be used if desired. Referring to FIG. 2, two third chambers 30 are respectively connected to the two upper outlets 24a of the second chamber 20 in a fluid communication manner, and one third chamber 30 is connected to one lower outlet 26a of the second chamber 20 in a fluid communication manner. Among the at least two third chambers 30, one may be used to contain a target material, and the other may be used to contain waste from which the target material is removed. In addition, the third chamber 30 may also be connected to another chamber in a fluid communication manner for a subsequent reaction.

The microfluidic device may control a flow of a fluid using a rotational force and a centrifugal force generated by rotating the body 1 and may mix or centrifuge two or more materials contained in the second chamber 20 by rotating the body 1 in the clockwise direction and/or the counter-clockwise direction. For example, two or more materials contained in the second chamber 20 may be mixed by repeatedly rotating the body 1 in the clockwise direction and/or the counter-clockwise direction or centrifuged by continuously rotating the body 1 in one of the clockwise direction and the counter-clockwise direction using density difference to form two or more material layers. Meanwhile, the second chamber 20 may further include a stirring unit 60 for mixing two or more materials contained in the second chamber 20. The stirring unit 60 may accelerate the mixing reaction by repeating rotation of the body 1. Furthermore, the stirring unit 60 may be formed of a heat-generating or magnetic force-generating material to improve efficiency of the biochemical reaction.

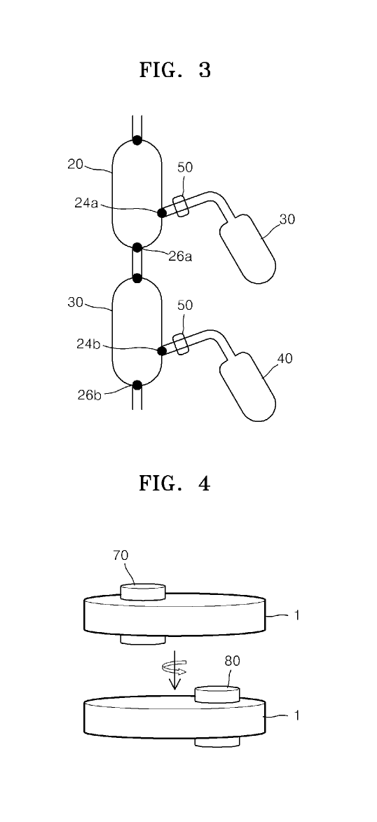

FIG. 3 shows a fourth chamber connected to a third chamber in a fluid communication manner in a microfluidic device according to an embodiment of the present disclosure.

The third chamber 30 may be connected to a fourth chamber 40 that is disposed to be spaced apart from the third chamber 30 in the centrifugal force direction in a fluid communication manner and includes at least one of an upper outlet and a lower outlet disposed to be spaced apart from the upper outlet in the centrifugal force direction. Referring to FIG. 3, the upper outlet 24a and the lower outlet 26a of the second chamber 20 are respectively connected to two third chambers 30 in a fluid communication manner. The third chamber 30 connected to the lower outlet 26a of the second chamber 20 may be connected to the fourth chamber 40 via an upper outlet 24b of the third chamber 30 in a fluid communication manner. Accordingly, the third chamber 30 of the microfluidic device may further include an upper outlet and/or a lower outlet connected to another chamber in a fluid communication manner when other products are generated from operations of the biochemical reaction. The third chamber 30 may further include an inlet for adding another sample and/or reagent, if desired. In this manner, any desired number of chambers may be connected to form a microfluidic device having a series of interconnected chambers spaced apart in a centrifugal force direction.

FIG. 4 shows a heater and a cooler of a microfluidic apparatus according to an embodiment of the present disclosure.

The microfluidic device according to the current embodiment may further include a heater 70 for heating the second chamber 20 and/or a cooler 80 for cooling the second chamber 20. The heater 70 and the cooler 80 may or may not contact the second chamber 20. For example, the heater 70 may be a contact type and may be a metallic heating block. Alternatively, the heater 70 may be a non-contact type and may be a device for providing electromagnetic waves, laser beams, or infrared rays. In this regard, the second chamber 20 of the body 1 may be formed of a light-transmitting material regardless of thermal conductivity. The heater 70 and the cooler 80 are used to perform a biochemical reaction including operations of heating and/or cooling a sample and/or a reagent accommodated in the second chamber 20. For example, the biochemical reaction may be a polymerase chain reaction (PCR), a widely used technique for amplifying a nucleic acid by geometric progression by repeating a process including denaturating a double-stranded deoxyribonucleic acid (DNA) to single-stranded DNA by heating a sample including the double-stranded DNA at about 95° C., providing forward and reverse oligonucleotide primers having sequences complementary to target sequences to be amplified, annealing the primers to the target sequences of the single-stranded DNA to form partial DNA-primer complexes by cooling the single-stranded DNA to about 55° C., and extending the double-stranded DNA using a DNA polymerase (e.g., Taq polymerase) based on the primers of the partial DNA-primer complexes by maintaining the annealed sample at about 72° C. As such, in a variety of biochemical reactions including PCR, applying heat to the sample and/or the reagent or taking heat out of the sample and/or the reagent, i.e., heating and cooling processes, may be repeated.

Referring to FIG. 4, heaters 70, for example two heaters 70 are disposed on upper and lower surfaces of the rotatable disc-type body 1, respectively, thereby covering the upper and lower surfaces of the rotatable disc-type body 1, in which the second chamber 20 (not shown) is disposed. Coolers 80, for examples two coolers 80 are disposed on upper and lower surfaces of the rotatable disc-type body 1, respectively, thereby covering the upper and lower surfaces of the rotatable disc-type body 1, in which the second chamber 20 (not shown) is disposed. The heaters 70 and coolers 80 may be disposed on the upper and lower surfaces of the rotatable disc-type body 1 in rotatable manner with each other. Thus, when the second chamber 20 of the body 1 is disposed between the heaters 70, the second chamber 20 may be locally heated. When the second chamber 20 is disposed between the coolers 80 by rotating the body 1, the second chamber 20 may be locally cooled. Thus, the microfluidic device may be used to perform various biochemical reactions including heating and/or cooling of reagents and samples. Additionally, the heaters 70 and coolers 80 may be disposed to cover or contact, as the case may be, any chamber, portion, surface, etc. of the rotatable disc-type body 1 in order to provide heating and/or cooling to the desired location.

A method of separating a target material according to an embodiment of the present disclosure includes providing a microfluidic device as described above, introducing at least one reagent to a first chamber, introducing a sample including a target material to the second chamber, transferring the reagent from the first chamber to the second chamber by rotating a body, mixing and reacting the reagent with the sample by repeatedly rotating the body in the clockwise direction and/or the counter-clockwise direction, centrifuging products of the reagent-sample reaction by continuously rotating the body in one of the clockwise and counter-clockwise directions, and separating the target material contained in an upper layer or lower layer of the second chamber via an upper outlet or a lower outlet of the second chamber.

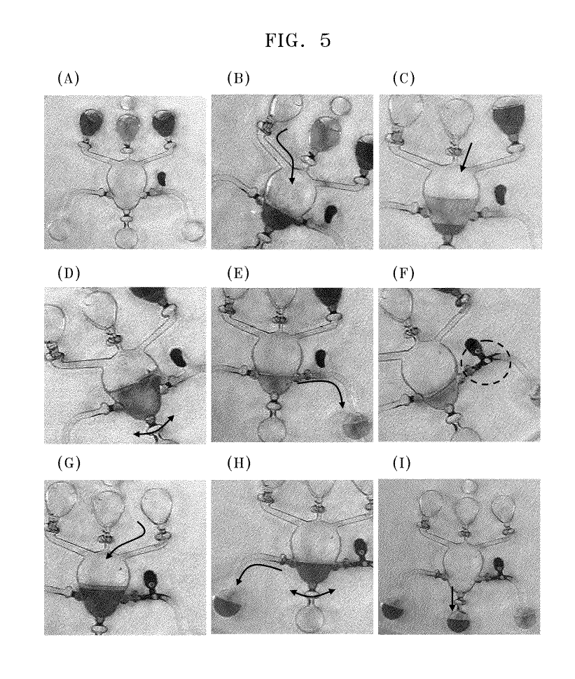

FIG. 5 shows a series of operations for controlling a transfer of a sample and a reagent in a microfluidic device according to an embodiment of the present disclosure. The example embodiment of FIG. 5 (A) illustrates providing a the microfluidic device, including first chambers filled with a sample and reagents. In this illustration, the microfluidic device includes three first chambers, a second chamber connected to the first chambers in a fluid communication manner, and three third chambers respectively connected to two upper outlets and one lower outlet of the second chamber in a fluid communication manner. FIG. 5(B) illustrates transferring the sample from one of the first chambers, initially including the sample, to the second chamber by opening a valve of the first chamber and rotating a body. FIG. 5(C) illustrates transferring of a first reagent from one of the first chambers, initially including the first reagent, to the second chamber by opening a valve of the first chamber and rotating the body. FIG. 5(D) illustrates mixing the sample with the first reagent by repeatedly rotating the body in the clockwise direction and the counter-clockwise direction. FIG. 5(E) illustrates transferring a material with a relatively low density (e.g., supernatant) contained in the mixture of the sample and the first reagent from the second chamber to one third chamber by opening a valve of a right upper outlet of the second chamber and rotating the body. FIG. 5(F) illustrates closing of the valve of the right upper outlet of the second chamber. FIG. 5(G) illustrates transferring a second reagent from one of the first chambers, initially including the second reagent, to the second chamber by opening a valve of the first chamber and rotating the body. FIG. 5(H) illustrates transferring a material with a relatively low density (e.g., supernatant) contained in the mixture of the sample and the second reagent from the second chamber to another third chamber by mixing the sample with the second reagent by repeatedly rotating the body in the clockwise direction and the counter-clockwise direction and opening a valve of a left upper outlet of the second chamber and rotating the body. FIG. 5(I) illustrates transferring a material with a relatively high density (lower layer, e.g., sediment) contained in the mixture of the sample and the second reagent from the second chamber to another third chamber by opening a valve of a lower outlet of the second chamber and rotating the body.

The microfluidic device may be used for various biochemical reactions including separating a target material from a mixture of two or more materials with different densities.

The microfluidic device may be used for a reaction extracting a nucleic acid from whole blood by using a biochemical reagent. In this example, the sample may include cells, and the reagent may include at least one reagent required to extract a genome from the cells. Particularly, a reaction for extracting a nucleic acid from the whole blood using biochemical reagents includes 1) separating white blood cells from the whole blood (first operation), 2) lysing cells by lysing cell membranes of white blood cells to discharge intracellular content (second operation), 3) purifying the nucleic acid (DNA) from the intracellular content (third operation), 4) concentrating the nucleic acid (fourth operation), 5) washing the resultant to remove residual salts (fifth operation), and 6) eluting the nucleic acid to stably store the nucleic acid (sixth operation). In this example, operations of mixing the reagent and the sample and centrifuging the mixture are required in the separating of the cells (first operation), the concentrating of the nucleic acid (fourth operation), and the washing (fifth operation). Thus, a plurality of reagents used for the nucleic acid extraction are added to a chambers of the microfluidic device as described above, the reagents and a whole blood sample are added to the second chamber, the reagents and the sample are mixed by repeatedly rotating the body of the microfluidic device in the clockwise direction and/or the counter-clockwise direction, and the products of the reaction are centrifuged by continuously rotating the body in one of the clockwise direction and the counter-clockwise direction, to separate the target material contained in a upper layer (for example, supernatant) or a lower layer (for example, a sediment) via the upper outlet or lower outlet of the second chamber.

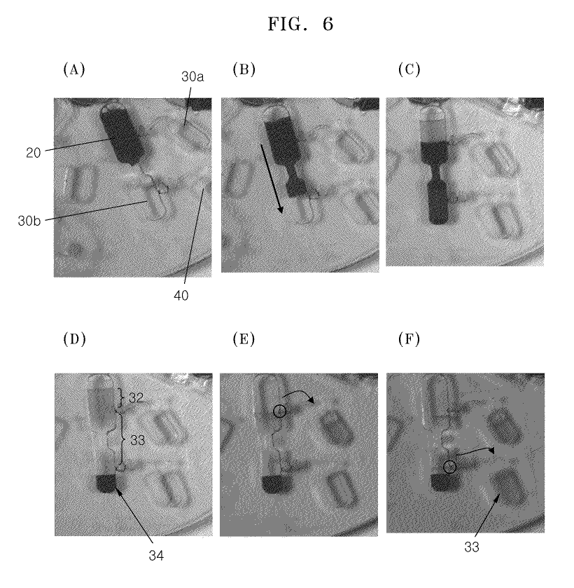

FIG. 6 illustrates a series of operations for separating white blood cells from a whole blood sample by using a microfluidic device according to an embodiment of the present disclosure. FIG. 6(A) illustrates a second chamber (20) of the microfluidic device including whole blood, wherein the microfluidic device includes a third chamber (30a) connected to an upper outlet of the second chamber, another third chamber (30b) connected to a lower outlet of the second chamber, and a fourth chamber (40) connected to an outlet of the latter third chamber (30b). The third chamber (30b) may include a density gradient medium. FIG. 6(B) illustrates transferring the whole blood to the third chamber (30b) by rotating the body of the microfluidic device (G-Force direction, see arrow). FIGS. 6(C)-(D) illustrate separating the mixture of the intracellular content into layers due to density difference to obtain products of the centrifugation by continuously rotating the body in one of the clockwise direction and the counter-clockwise direction. Particularly, products of the centrifugation include waste (32), white blood cells (WBC) (33), and red blood cells (RBC) (34) from the top layer. FIG. 6(E) illustrates transferring the waste (upper layer, for example, supernatant) to the third chamber by opening a valve of the upper outlet of the second chamber and rotating the body. FIG. 6(F) illustrates transferring the white blood cells (upper layer, for example, supernatant) to the fourth chamber by opening the outlet of the third chamber and rotating the body, while red blood cells (lower layer, for example, sediment) remain in the third chamber.

In addition, the microfluidic device may be used in an emulsion nucleic acid amplification. An emulsion nucleic acid amplification, e.g., an emulsion PCR, that is, a technique of amplifying a target region of a nucleic acid using a small amount of a genome is efficiently used to detect a mutation with high sensitivity. In this regard, the emulsion PCR may be efficiently performed in an automated single device by using the microfluidic device according to an embodiment of the present disclosure.

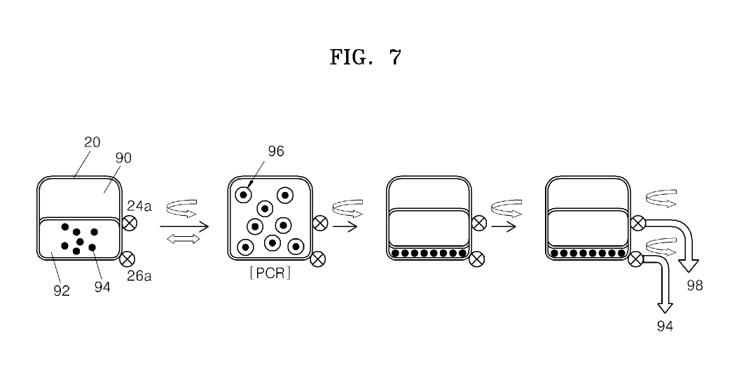

FIG. 7 shows a series of operations of a nucleic acid amplification technique in an emulsion conducted by using a microfluidic apparatus according to an embodiment of the present disclosure. A method of separating products of a nucleic acid amplification according to this example includes: providing the microfluidic device described above, introducing at least one reagent and oil used to perform a nucleic acid amplification to first chambers, introducing a sample including a template nucleic acid to a second chamber, transferring the reagent from one first chamber to the second chamber by rotating the body and mixing the reagent and the sample by repeatedly rotating the body in the clockwise direction and/or the counter-clockwise direction, transferring the oil from another first chamber to the second chamber by rotating the body and forming an emulsion in which the sample is surrounded by the oil by repeatedly rotating the body in the clockwise direction and/or the counter-clockwise direction, amplifying a nucleic acid of the sample in the emulsion by heating and/or cooling the second chamber, centrifuging the resultant by continuously rotating the body in one of the clockwise direction and the counter-clockwise direction, and separating products of the amplification contained in an upper layer (e.g., supernatant) or a lower layer (e.g., a sediment) via an upper outlet or a lower outlet of the second chamber.

Referring to FIG. 7, a first operation shows that the oil (90) having a relatively low density and an aqueous solution (92) including the reagent and the sample and having a relatively high density respectively form the upper layer (e.g., supernatant) and the lower layer (e.g., sediment) in the second chamber. In addition, a second operation shows formation of a water/oil emulsion (96) in which the sample is surrounded by the oil by transferring the oil from the first chamber to the second chamber by rotating the body and repeatedly rotating the body in the clockwise direction and/or the counter-clockwise direction, and a nucleic acid amplification including a denaturation step of the sample of the emulsion performed at about 95° C., an annealing step performed at about 55° C., and an extension step performed at about 72° C. by heating and/or cooling the second chamber. In addition, a third operation shows centrifugation performed by continuously rotating the body in the clockwise direction or the counter-clockwise direction, and results of the centrifugation. The last fourth operation shows separation of products (94) of the nucleic acid amplification by separating an upper layer (e.g., supernatant, waste 98) and a lower layer (e.g., sediment) from the second chamber via an upper outlet and a lower outlet by rotating the body.

As described above, a target material may be efficiently separated from a mixture including two or more materials with different densities and an emulsion nucleic acid amplification may also be efficiently conducted by using a microfluidic device according to one or more of the above embodiments of the present disclosure.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A microfluidic device for separating a target material from a mixture of materials having different densities using a centrifugal force and a rotational force, the device comprising:
a rotatable disc-type body having a center;
a first chamber disposed in or on the body and spaced apart from the center in a centrifugal force direction;
a second chamber disposed in or on the body and spaced apart from the first chamber in the centrifugal force direction and in fluid communication with the first chamber, the second chamber comprising an upper outlet and a lower outlet spaced apart from the upper outlet in the centrifugal force direction; and
a plurality of third chambers disposed in or on the body, wherein each of the upper outlet and the lower outlet of the second chamber is in fluid communication with at least one of the third chambers.

2. The microfluidic device of claim 1, wherein the device comprises valves disposed between the first chamber and the second chamber and between the second chamber and the third chamber to control fluid flow.

3. The microfluidic device of claim 2, wherein at least one valve is a phase transition type valve.

4. The microfluidic device of claim 1, wherein the second chamber further comprises a stirring unit for mixing two or more materials accommodated in the second chamber.

5. The microfluidic device of claim 1, wherein at least one third chamber is in fluid communication with a fourth chamber that is spaced apart from the third chamber in the centrifugal force direction, and wherein the fourth chamber comprises at least one of an upper outlet and a lower outlet spaced apart from the upper outlet in the centrifugal force direction.

6. The microfluidic device of claim 1, further comprising a contact type or non-contact type heater disposed to heat the second chamber, a contact type or non-contact type cooler disposed to cool the second chamber, or both.

7. The microfluidic device of claim 1, wherein the first chamber comprises at least one reagent used to extract a genome from a cell or a reagent used to amplify a target region of a genome.

8. A method for separating a target material, the method comprising:
providing the microfluidic device of claim 1;
introducing at least one reagent to the first chamber;
introducing a sample comprising a target material to the second chamber;
rotating the body, thereby transferring the reagent from the first chamber to the second chamber and mixing and reacting the reagent with the sample, wherein the body is optionally rotated sequentially in a clockwise and counterclockwise direction;
centrifuging products of the reagent-sample reaction by continuously rotating the body; and
separating the target material contained in a upper layer or lower layer of the second chamber via an upper outlet or a lower outlet of the second chamber.

9. The method of claim 8, wherein the sample comprises a cell, and the reagent comprises at least one reagent used to extract a genome from the cell.

10. The method of claim 8, wherein valves are disposed between the first chamber and the second chamber and between the second chamber and the third chamber to control fluid flow in the microfluidic device.

11. The method of claim 10, wherein the valve is a phase transition type valve.

12. The method of claim 8, wherein the second chamber further comprises a stirring unit for mixing two or more materials accommodated in the second chamber.

13. The method of claim 8, wherein at least one third chamber is connected to and in fluid communication with a fourth chamber that is spaced apart from the third chamber in the centrifugal force direction and comprises at least one of an upper outlet and a lower outlet spaced apart from the upper outlet in the centrifugal force direction.

14. The method of claim 8, further comprising a contact type or non-contact type heater disposed to heat the second chamber and/or a contact type or non-contact type cooler disposed to cool the second chamber.

15. The method of claim 8, wherein the first chamber comprises at least one reagent used to extract a genome from a cell or a reagent used to amplify a target region of a genome.

16. A method for amplifying a nucleic acid, the method comprising:
providing a microfluidic device of claim 23;
introducing at least one nucleic acid amplification reagent to at least one of the plurality of first chambers of the device, and oil to at least one other of the plurality of first chambers of the device,
introducing a sample including a template nucleic acid to a second chamber of the device; and
rotating the body of the device, thereby transferring the reagent from one first chamber to the second chamber and mixing the reagent with the sample;
and transferring the oil from another first chamber to the second chamber and forming an emulsion in which the sample is surrounded by the oil, wherein the body of the device is optionally rotated sequentially in a clockwise and counterclockwise direction;
amplifying a nucleic acid of the sample in the emulsion by heating and/or cooling the second chamber;
centrifuging a resultant by continuously rotating the body; and
separating products of the amplification contained in a supernatant or a sediment via an upper outlet or a lower outlet of the second chamber.

17. The method of claim 16, wherein at least one valve is disposed between the first chamber and the second chamber, and between the second chamber and the third chamber to control fluid flow in the microfluidic device.

18. The method of claim 17, wherein at least one valve is a phase transition type valve.

19. The method of claim 16, wherein the second chamber further comprises a stirring unit for mixing two or more materials accommodated in the second chamber.

20. The method of claim 16, wherein at least one third chamber is in fluid communication with a fourth chamber that is spaced apart from the third chamber in the centrifugal force direction and comprises at least one of an upper outlet and a lower outlet spaced apart from the upper outlet in the centrifugal force direction.

21. The method of claim 16, further comprising a contact type or non-contact type heater disposed to heat the second chamber, a contact type or non-contact type cooler disposed to cool the second chamber, or both.

22. The method of claim 16, wherein at least one first chamber comprises at least one reagent used to extract a genome from a cell or a reagent used to amplify a target region of a genome.

23. The device of claim 1, wherein the device comprises a plurality of first chambers in fluid communication with one or more second chambers.

24. The device of claim 1, wherein the device comprises a plurality of second chambers, and each of the second chambers is in fluid communication with one or more first chambers.
